(19) United States
(12) Patent Application Publication
(10) Pub. No.: US 2022/0285023 A1
(43) Pub. Date: Sep. 8, 2022

(54) BILIARY ATRESIA DIAGNOSIS SUPPORT MANAGEMENT SYSTEM

(71) Applicants: SEKISUI MEDICAL CO., LTD., Tokyo (JP); NATIONAL CENTER FOR CHILD HEALTH AND DEVELOPMENT, Tokyo (JP)

(72) Inventors: Takeshi URAMOTO, Tokyo (JP); Tetsuaki KON, Tokyo (JP); Tomoyoshi SHIROSHITA, Tokyo (JP); Akira MATSUI, DECEASED (US); Hideki NAKAJIMA, Tokyo (JP); Hiroyuki ABE, Tokyo (JP)

(73) Assignees: SEKISUI MEDICAL CO., LTD., Tokyo (JP); NATIONAL CENTER FOR CHILD HEALTH AND DEVELOPMENT, Tokyo (JP)

(21) Appl. No.: 17/635,042

(22) PCT Filed: Aug. 13, 2020
(86) PCT No.: PCT/JP2020/030814

§ 371 Date: Feb. 14, 2022

(30) Foreign Application Priority Data
Aug. 15, 2019 (JP) 2019-149008

(51) Int. Cl. G16H 40/67; G16H 30/20

(52) U.S. Cl. CPC G16H 40/67; G16H 30/20

(57) ABSTRACT

There is provided a biliary atresia diagnosis support management system including a server, the server including a transmission/reception unit receiving an image of stool from a living body and a determination unit determining whether biliary atresia is contracted or not from the image, wherein the server further includes: a management unit including a usage fee saving unit saving received usage fees and a donation sending unit for sending a donation from the saved usage fees; and a doctor's determination request processing unit requesting a determination by a doctor if the determination unit determines that there is a suspected positive result; and if it is diagnosed that the living body needs treatment for biliary atresia as a result of a thorough examination, the server pays a donation for the treatment from the usage fees saved in the usage fee saving unit via the donation sending unit. According to such a system, not only a determination about biliary atresia is made by software, but also a determination and a thorough examination by a doctor, and treatment after that are supported.

### TECHNICAL FIELD

The present invention relates to a biliary atresia diagnosis support management system. The present invention also relates to an application program for biliary atresia diagnosis.

### BACKGROUND ART

Biliary atresia is such an intractable disease that a tube called the bile duct that connects the liver and the duodenum is blocked congenitally or soon after birth, and thereby, bile cannot be released from the liver into the intestines. Though biliary atresia is a rare disease that one baby among 9,000 births contracts, the mortality is the highest among liver/biliary tract diseases of the same age.

Light yellow stool is one of the most specific symptoms of biliary atresia. For 70 to 80% of biliary atresia patient babies, the stool color changes from yellowish to light yellow by four weeks after birth, and, for most of the remaining 20 to 30%, the light yellow stool appears by two months after birth. In Japan, Stool Color Cards have been introduced nationwide for biliary atresia screening since 2012. In the United States, a smartphone application for the purpose of biliary atresia screening exists (Non Patent Literature 1).

### CITATION LIST

### Non Patent Literature

Non Patent Literature 1: Franciscovich, Amy, et al. “PoopMD, a Mobile Health Application, Accurately Identifies Infant Acholic Stools.” PloS one 10.7 (2015): e0132270

### SUMMARY OF INVENTION

### Technical Problem

In the technique disclosed in Non Patent Literature 1, though biliary atresia diagnosis by software is possible, there is a possibility that an erroneous determination occurs because a biliary atresia diagnosis by a doctor is not made. Further, it is conceivable that, when a positive result for biliary atresia is determined, it may be difficult for a user to judge which medical institution he should visit to receive a definitive diagnosis. Further, though an operation to release the blockage of the bile duct is given as a treatment for biliary atresia, it is necessary to perform a liver transplant if jaundice is not improved by the operation. There is also a need for a method for relieving the burden of treatment costs in that case.

An object of the present invention is to provide a biliary atresia diagnosis support management system not only for making a determination of biliary atresia by software but also for supporting a determination and a thorough examination by a doctor and treatment after that, and an application program for biliary atresia diagnosis.

### Solution to Problem

Specifically, the present invention is as follows.

<1>

A biliary atresia diagnosis support management system including a server, the server including a transmission/reception unit receiving an image of stool from a living body and a determination unit determining whether biliary atresia is contracted or not from the image, wherein

the server further includes:

a management unit including a usage fee saving unit saving received usage fees and a donation sending unit for sending a donation from the saved usage fees; and

a doctor's determination request processing unit requesting a determination by a doctor if the determination unit determines that there is a suspected positive result; and

if it is diagnosed that the living body needs treatment for biliary atresia as a result of a thorough examination, the server pays a donation for the treatment from the usage fees saved in the usage fee saving unit via the donation sending unit.

<2>

The biliary atresia diagnosis support management system according to <1>, further including a first terminal including a transmission/reception unit transmitting an image of stool from a living body and receiving a diagnosis result via the server, a usage fee sending unit for paying the usage fee and a donation reception unit for receiving a donation.

<3>

The biliary atresia diagnosis support management system according to <2>, wherein the first terminal further includes a disease information reception unit including at least one means among a determination result display unit, a thorough examination guide display unit and a treatment policy display unit.

<4>

The biliary atresia diagnosis support management system according to <2> or <3>, further including a second terminal transmitting at least any one of a determination result by the determination unit and a determination result by a doctor to at least any one of the first terminal and the server, wherein

if the determination unit determines that there is a suspected positive result, the second terminal transmits at least any one of the determination result by the doctor and a thorough examination guide to at least any one of the server and the first terminal.

<5>

A biliary atresia diagnosis support management system including:

(A) a first terminal for a user to transmit an image of stool of a newborn baby or an infant to a server using an application;

(B) a server receiving the image from the first terminal; and

(C) a second terminal transmitting at least any one of a determination result by a determination unit and a determination result by a doctor to at least any one of the first terminal and the server; wherein

the first terminal includes a usage fee sending unit for the user of the application to pay a usage fee;

the server includes:

a determination unit determining whether biliary atresia is contracted or not from the image;

a usage fee saving unit saving received usage fees; and

a donation sending unit for sending a donation from the saved usage fees;

if the determination unit determines that there is a suspected positive result, the server transmits a request for a determination by a doctor together with the determination result to the second terminal via the server;

if the determination unit determines that there is a suspected positive result, the second terminal transmits at least any one of the determination result by the doctor and a thorough examination guide to at least any one of the server and the first terminal; and

if it is diagnosed that the newborn baby or the infant needs treatment for biliary atresia as a result of a thorough examination, the server pays a donation for the treatment from the usage fees saved in the usage fee saving unit via the donation sending unit.

<6>

The biliary atresia diagnosis support management system according to <5>, further including (D) thorough examination means for examining whether biliary atresia is contracted or not, wherein the thorough examination means performs the thorough examination about whether the newborn baby or the infant has contracted biliary atresia or not.

<7>

The biliary atresia diagnosis support management system according to <5> or <6>, wherein the first terminal further includes a donation request unit for the user to request the donation.

<8>

The biliary atresia diagnosis support management system according to any of <5> to <7>, wherein, if the determination unit determines that biliary atresia is negative, the determination result of being negative is transmitted to the first terminal.

<9>

The biliary atresia diagnosis support management system according to any of <5> to <8>, wherein

the determination unit further includes a change analysis unit;

the change analysis unit analyzes a change between an image of stool of the newborn baby or the infant transmitted before and the image of stool of the newborn baby or the infant newly transmitted; and

the determination unit determines whether biliary atresia is contracted or not based on a result of the analysis.

<10>

The biliary atresia diagnosis support management system according to any of <5> to <9>, wherein the first terminal further includes a stool information input unit for transmitting stool information.

<11>

The biliary atresia diagnosis support management system according to any of <5> to <10>, wherein the second terminal includes an information request unit requesting further information from the server.

<12>

An application program for biliary atresia diagnosis to be installed on a terminal that is capable of transmitting an image of stool of a newborn baby or an infant to a server, the application program including:

means for a user of an application to send a usage fee;

means for causing information about a newborn baby or an infant to be inputted;

means for causing an image of stool of the newborn baby or the infant to be inputted;

means for receiving a guide for a thorough examination for examining whether biliary atresia is contracted or not;

means for displaying information about a determination result about a biliary atresia diagnosis received from the server or a doctor; and

means for receiving a donation for treatment for biliary atresia.

### Advantageous Effect of Invention

In the present invention, if a determination unit of a server determines that there is a suspected positive result, a determination request to a doctor is notified to a second terminal from the server, and a determination is made by the doctor. Therefore, a more certain determination result can be ensured. Further, in the present invention, if it is determined by the doctor that there is a suspected positive result, a guide for a thorough examination by thorough examination means is notified to a first terminal that a user has. Additionally, if it is diagnosed that treatment for biliary atresia is necessary for a newborn baby or an infant as a result of the thorough examination by the thorough examination means, a donation for the treatment is paid from usage fees saved in a usage fee storage unit, so that a financial burden on the user can be relieved.

### BRIEF DESCRIPTION OF DRAWINGS

FIG. 1 is a diagram for illustrating a configuration of a biliary atresia diagnosis support management system according to an embodiment.

FIG. 2 is a diagram for illustrating a specific example of the biliary atresia diagnosis support management system of the present invention.

FIG. 3 is a diagram for illustrating a functional configuration of a first terminal.

FIG. 4 FIGS. 4A to 4C are diagrams showing screen examples of an application for terminal displayed on the first terminal, respectively.

FIG. 5 FIGS. 5A to 5D are diagrams showing screen examples for disease information displayed by the first terminal, respectively.

FIG. 6 is a diagram for illustrating a functional configuration of a server.

FIG. 7 is a diagram for illustrating a functional configuration of a second terminal.

FIG. 8 is a diagram for illustrating an operation of the biliary atresia diagnosis support management system of the present invention.

### DESCRIPTION OF EMBODIMENTS

Explanation of the present invention will be made below by giving an embodiment. The present invention, however, is not limited to the embodiment below. In drawings, those with the same or similar functions will be given the same or similar signs, and repeated explanation will be omitted. The drawings are schematic. Therefore, specific mechanisms and the like should be judged by checking in comparison with the explanation below. Here, the explanation will be made, with a newborn baby or an infant as an example of a living body.

### Biliary Atresia Diagnosis Support Management System

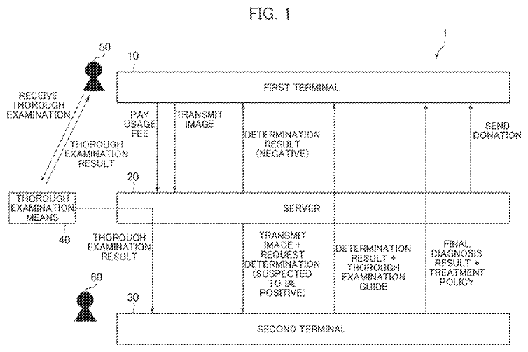

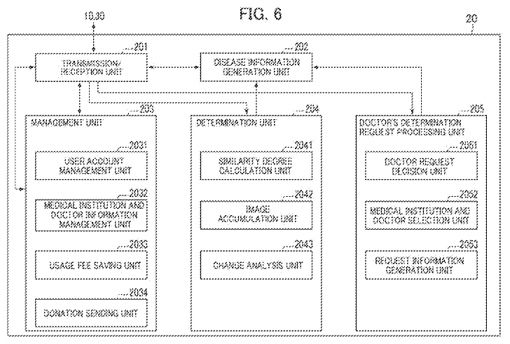

FIG. 1 is a diagram for illustrating a configuration of a biliary atresia diagnosis support management system 1 according to the present invention. The biliary atresia diagnosis support management system 1 is provided with a first terminal 10, a server 20, a second terminal 30 and thorough examination means 40. FIG. 6 is a diagram for illustrating a functional configuration of the server 20.

The present invention relates to the biliary atresia diagnosis support management system 1 provided with the server 20 provided with a transmission/reception unit 201 that receives an image of stool of a living body (a newborn baby or an infant) and a determination unit 204 that determines whether biliary atresia is contracted or not from the image. The server 20 further includes a management unit 203 including a usage fee saving unit 2033 that saves received usage fees and a donation sending unit 2034 for sending a donation from the saved usage fees; and a doctor's determination request processing unit 205 that requests a determination by a doctor if the determination unit 204 determines that there is a suspected positive result.

The biliary atresia diagnosis support management system 1 may be configured to be further provided with the first terminal 10 including a transmission/reception unit 101 that transmits an image of stool of a newborn baby or an infant and receives a diagnosis result via the server 20, a usage fee sending unit 103 for paying a usage fee and a donation reception unit 104 for receiving a donation. The first terminal 10 may be configured to be further provided with a disease information reception unit 106 including at least one means among a determination result display unit 1061, a thorough examination guide display unit 1062 and a treatment policy display unit 1063.

The biliary atresia diagnosis support management system 1 may be configured to be further provided with the second terminal 30 that transmits at least any one of a determination result by the determination unit 204 and a determination result by a doctor to at least any one of the first terminal 10 and the server 20.

The second terminal 30 may be configured to, if the determination unit 204 determines that there is a suspected positive result, transmit at least any one of the determination result by the doctor and a thorough examination guide to at least any one of the server 20 and the first terminal 10.

Next, specific functions of the biliary atresia diagnosis support management system 1 of FIG. 1 will be explained.

A user 50 pays an application usage fee using the first terminal 10. The user 50 transmits a photographed image of stool of a newborn baby or an infant to the server 20 using the first terminal 10.

In the server 20, the received image is analyzed by the determination unit 204. For example, the analysis is performed by calling reference stool images recorded in a recording portion (not shown) of the server 20 and analyzing a degree of similarity between the received image and the reference stool images. If there is a determination result of negative for biliary atresia as a result of the analysis, the transmission/reception unit 201 of the server 20 transmits information of being negative for biliary atresia to the first terminal 10 or transmits nothing. If it is determined that there is a suspected positive result for biliary atresia as a result of the analysis, the doctor's determination request processing unit 205 of the server 20 transmits a determination request to a doctor together with the stool image, to the second terminal 30. After that, image analysis is performed by a doctor 60.

The second terminal 30 transmits a determination result by the doctor 60 to the first terminal 10 via the server 20 or directly. If the determination result by the doctor 60 is that there is still a suspected positive result for biliary atresia, the second terminal 30 transmits a thorough examination guide explaining that a thorough examination is necessary together with the determination result, to the first terminal 10 via the server 20 or directly. If the determination result by the doctor 60 is “negative”, the second terminal 30 transmits information of being negative to the first terminal 10 or transmits nothing. The patient receives a thorough examination at the thorough examination means 40.

The thorough examination means 40 performs the thorough examination and communicates a result of the thorough examination to the user 50. In this case, the user 50 can bring the result of the thorough examination to receive a final diagnosis by the doctor 60. In this case, a medical specialist enrolled in a thorough examination institution may make the final diagnosis. Further, the thorough examination means 40 performs the thorough examination and transmits the result of the thorough examination to the second terminal 30 via the server 20. The doctor 60 makes the final diagnosis referring to the thorough examination result and transmits a final diagnosis result to the first terminal 10 via the server 20 or directly.

If it is finally diagnosed that treatment for biliary atresia is necessary, as a result of the thorough examination, a donation is sent from the server 20 to the user 50 via the first terminal 10. Specifically, the donation for the treatment may be paid from the usage fees saved in the usage fee saving unit 2033 by the server 20 via the donation sending unit 2034.

### Specific Example of Biliary Atresia Diagnosis Support Management System

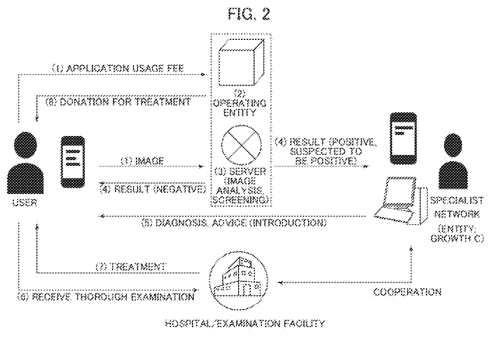

FIG. 2 is a diagram for illustrating a specific example of the biliary atresia diagnosis support management system 1 according to the present invention. A communication terminal machine is not especially limited, and, for example, a personal computer, a tablet terminal, a smartphone or the like can be used. Here, explanation will be made with a smartphone as an example.

A user pays an application usage fee to an application operating entity via a server using a smartphone. The user transmits a photographed image of stool of a newborn baby or an infant to the server using the smartphone. A determination unit of the server analyzes the received image. If a determination of being negative for biliary atresia is made as a result of the analysis, information of being negative for biliary atresia is transmitted to the smartphone of the user, or nothing is transmitted. If it is determined that there is a suspected positive result for biliary atresia as a result of the analysis, the server transmits a determination request together with the image of the stool to a computer beside a specialist. After that, analysis by the specialist is performed.

The computer beside the specialist transmits a determination result by the specialist to the smartphone of the user via the server or directly. If the determination result by the specialist is that there is still a suspected positive result for biliary atresia, a second terminal transmits a thorough examination guide explaining that a thorough examination is necessary together with the determination result, to a first terminal via the server or directly. The patient receives a thorough examination at a hospital or an examination facility.

If it is finally diagnosed that treatment for biliary atresia is necessary as a result of the thorough examination, a donation is sent from the server to the user via the first terminal.

### Functional Configuration of First Terminal

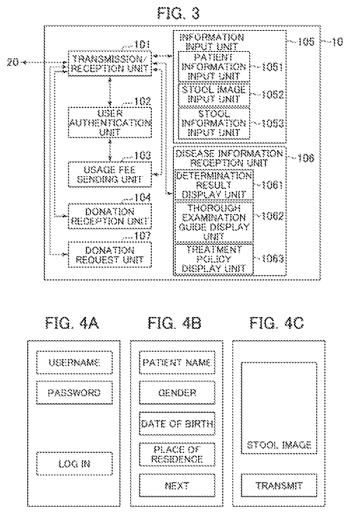

FIG. 3 is a diagram for illustrating a functional configuration of the first terminal 10. An application program has been installed in the first terminal 10, and it is possible to, by transmitting an image of stool of a newborn baby or an infant to the server 20, receive a result of determination about contraction of biliary atresia corresponding to the image of the stool from the server 20. The first terminal 10 includes the transmission/reception unit 101, a user authentication unit 102, the usage fee sending unit 103, the donation reception unit 104, an information input unit 105, the disease information reception unit 106 and a donation request unit 107.

The information input unit 105 is provided with a patient information input unit 1051, a stool image input unit 1052 and a stool information input unit 1053.

The disease information reception unit 106 is provided with the determination result display unit 1061, the thorough examination guide display unit 1062 and the treatment policy display unit 1063.

These components are realized by a CPU (central processing unit), a GPU (graphics processing unit), a memory, a program loaded to the memory and the like of a computer as hardware components. Here, functional blocks realized by cooperation among them are shown. Therefore, one skilled in the art would understand that these functional blocks can be realized in various forms only by hardware, only by software or by a combination of hardware and software.

The user authentication unit 102 authenticates whether a username and a password inputted by the user 50 are the same as user account data stored in the server 20. If the user 50 does not have a user account, the user authentication unit 102 displays a screen requesting creation of a user account. At the time creating a user account, the user authentication unit 102 creates the user account by causing a mail address, the user's account name, a password, a date of birth of a newborn baby or an infant, a gender of the newborn baby or the infant, whether the newborn baby or the infant has a disease or not, and the like to be inputted. The inputted information about the user 50 is stored as the user information into the recording unit of the server 20 in association with a user account ID.

The usage fee sending unit 103 pays a monthly usage fee to the operating entity via the server 20. Personal information required to send the usage fee, such as the address, account number and credit card number of the user 50, may be stored in the server 20 as user account data as necessary.

The donation reception unit 104 displays that a donation has been received for a target determined to need treatment for biliary atresia as a result of a thorough examination by the thorough examination means 40. A setting can be made so that the donation is received at a time or periodically received, for example, every six months. Personal information required to receive the donation, such as the address and account number of the user 50, may be stored in the server 20 as the user account data as necessary.

The patient information input unit 1051 notifies the user 50 to input a patient name, a gender of the patient, the date of birth of the patient, a place of residence of the patient and a medical history of the patient as information about the newborn baby or the infant who is the patient. The stool image input unit 1052 causes a photographed image of stool to be inputted. The stool image input unit 1052 displays a screen to cause a plurality kinds of stool images, such as a stool image photographed so that all the stool can be seen and an enlarged stool image photographed at a position close to the stool, to be inputted. On the stool information input unit 1053, information about defecation time and the like can be inputted together with the images as necessary.

The disease information reception unit 106 receives the information corresponding to the transmitted stool images from the server 20 via the transmission/reception unit 101 and displays the information to the user 50. The determination result display unit 1061 displays a determination result by the determination unit 204 of the server 20 and a determination result by the doctor 60 on the screen. Further, in the case of a final diagnosis by the doctor 60 being transmitted to the first terminal 10, the determination result display unit 1061 displays a final diagnosis result in consideration of a result of a thorough examination by the thorough examination means 40, which has been received from the server 20 or the second terminal 30, on the screen. The thorough examination guide display unit 1062 displays a thorough examination guide. If the thorough examination guide transmitted from the second terminal 30 via the server 20 or directly includes information about a medical institution or an examination institution specified by the doctor 60, the thorough examination guide display unit 1062 displays the medical institution or the examination institution on the screen. The treatment policy display unit 1063 displays an outline of a treatment policy for treatment for biliary atresia. Further, the treatment policy display unit 1063 can be provided with an inquiry form for applying for counseling by the doctor 60, making a question and the like and can be set to be capable of transmitting the application, the question or the like to the server 20 via the transmission/reception unit 101.

If it is finally diagnosed that treatment for biliary atresia is necessary as a result of a thorough examination, the donation request unit 107 requests the server 20 to pay a donation. Information about a medical institution where and a date when the final diagnosis was made, a name of the doctor 60 who made the final diagnosis and the like can be inputted to the donation request unit 107. It is also possible for the doctor 60 who made the final diagnosis to input a user-specific code to transmit the code to the server 20 so that authentication is performed with the request for the donation from the user and the code. Further, it is also possible to make such a setting that, by the doctor 60 who made the final diagnosis inputting that the final diagnosis of the patient is “positive” to the server 20, the donation is automatically paid without the request for the donation from the user.

The first terminal 10 can be also provided with an advertisement display unit that displays an advertisement according to patient information.

The functional configuration of the first terminal described above will be specifically explained below through explanation of screen examples of the application for terminal.

### Screen Examples of Application for Terminal

FIGS. 4A to 4C are diagrams showing screen examples of the application for terminal displayed on the first terminal 10. The user 50 has a user account that identifies the user 50 himself. The user 50 signs in a biliary atresia diagnosis support service provided by the application program from a screen shown in FIG. 4A using the user account. Then, the user authentication unit 102 in FIG. 3 authenticates whether a username and a password are the same as user account data stored in the recording unit of the server 20. If the sign in is successful, the first terminal 10 proceeds to a screen for receiving a diagnosis.

As shown in FIG. 4B, the screen is switched to a patient information input screen. Then, on the patient information input screen, the user 50 causes the patient information input unit 1051 of the information input unit 105 to operate and inputs patient information. The user 50 touches “NEXT”.

As shown in FIG. 4C, the screen is switched to a stool image input screen. Then, the user 50 causes the stool image input unit 1052 of the information input unit 105 in FIG. 3 to operate and causes photographed images of stool to be inputted. The user 50 inputs stool image stored in the first terminal 10. After that, the user 50 touches “TRANSMIT”. As necessary, the user 50 can also cause the stool information input unit 1053 in FIG. 3 to operate and input information about defecation time and the like.

When the user 50 touches “TRANSMIT”, the transmission/reception unit 101 in FIG. 3 transmits the patient information and stool image that the user 50 has inputted, to the server 20. Thereby, the biliary atresia diagnosis support request process by the user 50 ends on the first terminal 10.

After that, in the server 20, determination about whether biliary atresia is contracted or not is executed based on the patient information and the stool images. The above determination about whether biliary atresia is contracted or not is performed, for example, by analyzing a degree of similarity with the reference stool images.

After the user 50 ends the biliary atresia diagnosis support request, a result of the determination about whether biliary atresia is contracted or not by the image analysis process is transmitted to the first terminal 10 from the server 20. It is also possible to make a setting so that nothing is transmitted in the case of being negative.

### Screen Examples for Biliary Atresia Diagnosis Support Information

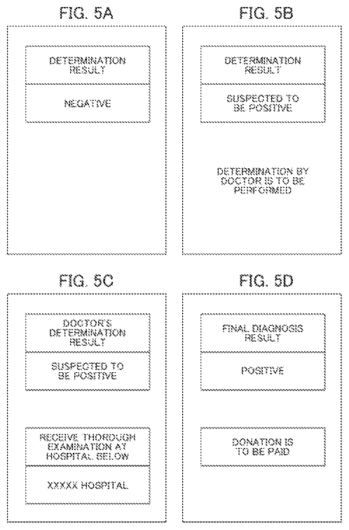

FIGS. 5A to 5D are diagrams showing screen examples for biliary atresia diagnosis support information that the first terminal 10 receives. If the determination result is “negative”, the determination result display unit 1061 of the first terminal 10 in FIG. 3 receives the determination result from the server 20 or the second terminal 30 via the transmission/reception unit 101. Then, as shown in FIG. 5A, the determination result of being negative is displayed on the screen.

It is also possible to make such a setting that, when a determination result shows being negative, nothing is displayed on the determination result display unit 1061.

FIG. 5B shows a screen example when the determination result is that there is a suspected positive result for biliary atresia. If the determination result is that there is a suspected positive result for biliary atresia, the first terminal 10 receives the determination result from the server 20 via the transmission/reception unit 101. Then, as shown in FIG. 5B, it is displayed on the screen that there is a suspected positive result for biliary atresia and that a determination by a doctor is to be made.

FIG. 5C shows a screen example when a determination result by the doctor 60 is that there is a suspected positive result for biliary atresia. If the determination result by the doctor 60 is still that there is a suspected positive result for biliary atresia, the determination result display unit 1061 and the thorough examination guide display unit 1062 receive the determination result and a thorough examination guide from the server 20 or the second terminal 30 via the transmission/reception unit 101. Then, as shown in FIG. 5C, it is displayed on the screen that there is a suspected positive result, together with a guide for receiving a thorough examination.

In the case of “negative” as a result of the diagnosis by the doctor 60, information of being negative is displayed on the screen as shown in FIG. 5A.

FIG. 5D shows a screen example when a final diagnosis result in consideration of a result of the thorough examination is positive for biliary atresia in the case of the final diagnosis by the doctor 60 being transmitted to the first terminal 10. When the final diagnosis result in consideration of the result of the thorough examination is positive for biliary atresia, the final diagnosis result of positive for biliary atresia is received from the server 20 or the second terminal 30 via the transmission/reception unit 101. Then, as shown in FIG. 5D, it is displayed on the screen that a donation is to be paid via the donation reception unit 104. If the final diagnosis result in consideration of the result of the thorough examination is “negative”, information of being negative is displayed on the screen.

### Functional Configuration of Server

FIG. 6 is a diagram for illustrating the functional configuration of the server 20. The server 20 includes the transmission/reception unit 201, a disease information generation unit 202, the management unit 203, the determination unit 204 and the doctor's determination request processing unit 205.

The management unit 203 includes a user account management unit 2031, a medical institution and doctor information management unit 2032, the usage fee saving unit 2033 and the donation sending unit 2034.

The determination unit 204 includes a similarity degree calculation unit 2041, an image accumulation unit 2042 and a change analysis unit 2043.

The doctor's determination request processing unit 205 includes a doctor request decision unit 2051, a medical institution and doctor selection unit 2052 and a request information generation unit 2053.

These components are realized by a CPU, a GPU, a memory, a program loaded to the memory and the like of a computer as hardware components.

The management unit 203 manages users 50 who hold the first terminals 10 and medical institutions and/or doctors 60 that hold the second terminals 30. At the time of signing in any of the first terminals 10 or second terminals 30, the management unit 203 compares a username and a password transmitted from the first terminal 10 or the second terminal 30 with user information stored in the user account management unit 2031 or medical institution and doctor information stored in the medical institution and doctor information management unit 2032, and transmits a result of the comparison to the first terminal 10 or the second terminal 30. The user account management unit 2031 manages usage fee payment histories and donation payment histories of the users 50. The user account management unit 2031 can also mark patients who are suspected to be positive by the determination unit 204 and/or the doctors 60 and, when a donation is requested, compare the marks and an account from which the donation has been requested.

The medical institution and doctor information management unit 2032 stores account information about the medical institutions and the doctors 60 that hold the second terminals 30. The usage fee saving unit 2033 saves usage fees sent from the first terminals 10; and, if a final diagnosis result in consideration of a result of a thorough examination is “positive”, a donation for treatment is sent to a relevant user 50 from the donation sending unit 2034. The donation sending unit 2034 can also send the donation to the user 50 in response to a request by the donation request unit 107 of the first terminal 10. If a result that the final diagnosis is “positive” has been transmitted to the server 20 from a second terminal 30 or thorough examination means 40, the donation sending unit 2034 can send the donation to the user 50 without the request from the donation request unit 107 of the first terminal 10.

The determination unit 204 determines whether biliary atresia is contracted or not based on acquired patient information and stool images.

The similarity degree calculation unit 2041 analyzes stool images transmitted from any of the first terminals 10 to analyze a degree of similarity with the reference stool images. The image accumulation unit 2042 stores the stool images transmitted from the first terminal 10. The stored stool images are used for analysis such as comparison with newly transmitted stool images, as accumulated stool image information. The change analysis unit 2043 analyzes changes in stool color in the transmitted stool images based on the accumulated stool image information.

It is also possible to use a publicly known application having the function of the determination unit 204. For example, an application described in Patent Literature 1 can be used.

If a determination result of the determination unit 204 is that there is a suspected positive result, the doctor's determination request processing unit 205 request a determination by a doctor 60 to a second terminal 30. The doctor's determination request processing unit 205 transmits stool images and patient information required for a diagnosis that have been transmitted from a first terminal 10 to the second terminal 30 via the transmission/reception unit 201. The doctor's determination request processing unit 205 is provided with the doctor request decision unit 2051, the medical institution and doctor selection unit 2052 and the request information generation unit 2053. The doctor request decision unit 2051 decides execution of diagnosis support by a doctor 60 when the determination result of the determination unit 204 is that there is a suspected positive result, and does not decide execution of the diagnosis support if the determination result of the determination unit 204 is “negative”.

When the determination result of the determination unit 204 is that there is a suspected positive result, the medical institution and doctor selection unit 2052 selects the second terminal 30 of any of the plurality of doctors 60 and requests a determination.

The request information generation unit 2053 generates request information and a request ID to be transmitted to the second terminal 30, from the stool images and patient information transmitted from the first terminal 10. The request information is patient information from which personal information about a patient is excluded and includes the stool images and the patient information required for a diagnosis (the date of birth, a gender and the like) that have been transmitted from the first terminal 10. The request ID includes information about the first terminal 10, information about the second terminal 30 and a request identification number. The request information generation unit 2053 transmits the request information to the second terminal 30 and sends the request ID to the disease information generation unit 202. Based on the request ID, the disease information generation unit 202 can transmit a diagnosis result transmitted from the second terminal 30 to the first terminal 10 corresponding to the request ID.

When receiving a determination result from the determination unit 204, the disease information generation unit 202 generates information to be transmitted to a relevant first terminal 10. When receiving a determination result from a second terminal 30, the disease information generation unit 202 generates information to be transmitted to the first terminal 10, such as a guide for thorough examination means and the determination result. The disease information generation unit 202 transmits the determination result and the like corresponding to stool images transmitted from the first terminal 10 to the first terminal 10 via the transmission/reception unit 201. The disease information generation unit 202 can add details (analysis results of a degree of similarity, a change and the like) of the determination result of the determination unit 204 to the information to be transmitted to the first terminal 10.

Thus, the server 20 can return information about stool images transmitted from each first terminal 10 to the first terminals 10.

If a doctor 60 judges that more information is necessary, and a request for the necessary information is given from an information request unit 305 of the second terminal 30, the disease information generation unit 202 generates information to be transmitted to the second terminal 30. The information to be transmitted to the second terminal 30 includes previous stool images of the patient for whom the determination was accepted, stool images of patients other than the patient for whom the determination was accepted, and the like.

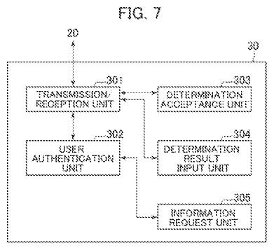

FIG. 7 is a diagram for illustrating a functional configuration of the second terminal 30. The application program for the biliary atresia diagnosis support management system 1 has been installed in the second terminal 30. The second terminal 30 includes a transmission/reception unit 301, a user authentication unit 302, a determination acceptance unit 303, a determination result input unit 304 and an information request unit 305.

These components are realized by a CPU, a GPU, a memory, a program loaded to the memory and the like of a computer as hardware components.

The user authentication unit 302 authenticates whether a username and a password are the same as medical institution or doctor account data stored in the server 20. The user authentication unit 302 may use biometric authentication such as fingerprint authentication or palm vein authentication at the time of login in order to confirm about being a relevant doctor 60, and personal information required for the biometric authentication may be stored in the server 20. Thus, in user authentication of the second terminal 30, security is higher than user authentication of each first terminal 10, and authentication requiring identity verification is executed.

The determination acceptance unit 303 receives a notification about whether it is possible to make a determination or not, from the server 20, and transmits whether or not it is accepted to make a determination, or that it is accepted to make a determination, to the server 20 via the transmission/reception unit 301. Receiving stool images and patient information transmitted from the server 20, the determination result input unit 304 inputs a determination result by a doctor 60, and the transmission/reception unit 301 transmits the determination result to the server 20. If the doctor 60 judges that more information is necessary, the doctor 60 can input necessary information to the information request unit 305 to request more information from the server 20. The necessary information includes previous stool images of the patient for whom the determination was accepted, stool images of patients other than the patient for whom a determination was accepted, and the like.

The thorough examination means 40 is arranged in a medical institution or examination institution capable of performing a blood test, a urine test, a duodenal fluid test, hepatobiliary scintigram and abdominal ultrasonography for diagnosing biliary atresia. The thorough examination means 40 includes a transmission unit 401 for transmitting a thorough examination result to the server 20 (not shown).

The thorough examination means 40 and a medical institution holding a second terminal 30 may be the same. That is, the medical institution holding a second terminal 30 may perform a thorough examination for biliary atresia for a patient as the thorough examination means 40. FIGS. 1, 2 and 8 show an aspect in which a medical institution holding the thorough examination means 40 and a medical institution holding the second terminal 30 are different.

In response to a request by a doctor 60, the thorough examination means 40 examines whether a patient has biliary atresia or not. When the thorough examination means 40 is connected to the server 20, the thorough examination means 40 transmits a result of the thorough examination to the doctor 60 via the server 20, and the doctor 60 makes a final diagnosis about whether biliary atresia is contracted or not, based on the thorough examination result, and transmits a final diagnosis result in consideration of the thorough examination result to a relevant first terminal 10 via the server 20 or directly. If the final diagnosis is “positive”, a treatment policy and the like are transmitted to the first terminal 10 together with the final diagnosis result in consideration of the thorough examination result.

Receiving the final diagnosis result of “positive”, the user 50 can request a donation from the server 20 via the donation request unit 107 of the first terminal 10. It is also possible for the donation sending unit 2034 of the server 20 automatically send the donation to the user 50 who received the final diagnosis result of “positive”. In the case of the user 50 requesting the donation from the server 20, it is also possible to compare a mark given to the patient determined to be suspected to be positive by the determination unit 204 and/or the doctor 60 with the account from which the donation was requested.

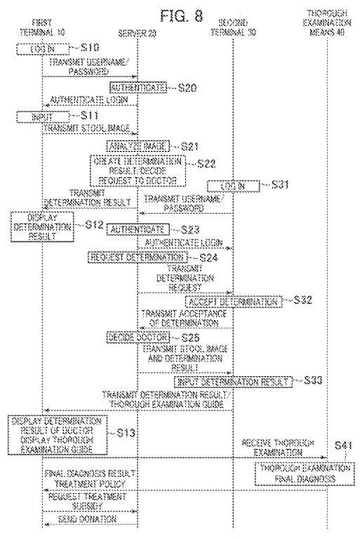

FIG. 8 is a diagram for illustrating an operation of the biliary atresia diagnosis support management system 1. Each user 50 monthly pays usage fees. The usage fees are saved in the server 20, and, if it is finally diagnosed that treatment for biliary atresia is necessary, a donation is sent to the first terminal 10 from pooled usage fees of all users.

First, the user 50 logs in the application program of the first terminal 10 (S10). The first terminal 10 transmits an inputted username and password to the server 20.

The server 20 compares the received username and password with a recorded user account (S20). If they correspond to the user account, the server 20 transmits information showing that the login is successful to the first terminal 10.

When the login is successful, stool images are inputted to the first terminal 10 by the user 50 (S11), and the stool images are transmitted to the server 20.

When receiving the stool images, the determination unit 204 of the server 20 executes an image analysis process (S21). The determination unit 204 determines whether biliary atresia is contracted or not. The disease information generation unit 202 generates information to be transmitted to the first terminal 10 (S22), and a determination result is transmitted to the first terminal 10.

The first terminal 10 displays the determination result received from the server 20 (S12). Thus, the user 50 of the first terminal 10 can acquire the determination result about whether biliary atresia is contracted or not in a short time.

An operation in a case where the determination result of the determination unit 204 of the server 20 is that there is a suspected positive result will be explained below.

If the determination result of the determination unit 204 is that there is a suspected positive result, the doctor's determination request processing unit 205 of the server 20 starts a determination request process and selects a doctor 60 (S22). The doctor's determination request processing unit 205 transmits information requesting a determination by the doctor 60 to a selected second terminal 30 via the transmission/reception unit of the server 20. The doctor's determination request processing unit 205 may transmit the request for a determination by the doctor 60 using a function of another application program, for example, a mail function.

The doctor 60 logs in the application program of the second terminal 30 (S31), and the second terminal 30 transmits an inputted username and password to the server 20. The server 20 compares the received username and password with stored doctor information (S23). If they correspond to the doctor information, the server 20 transmits information showing that the login is successful to the second terminal 30.

A determination request is transmitted to the doctor 60 who has succeeded in the login (S24). If the doctor 60 accepts to make a determination (S32), the second terminal 30 receives an input of accepting to make a determination by the doctor 60 (S32) and transmits information showing the acceptance to make a determination to the server 20. Thereby, the medical institution and doctor selection unit 2052 of the server 20 decides the medical institution and the doctor 60 (S25) and transmits the stool images and the determination result to the second terminal 30 that the medical institution and the doctor 60 hold.

The doctor 60 determines whether biliary atresia is contracted or not, based on the transmitted stool images, determination result and patient information. Receiving input of a determination result by the doctor 60 (S33), the second terminal 30 transmits the determination result by the doctor 60 to the first terminal 10. If the determination result by the doctor 60 is that there is a suspected positive result, the doctor 60 transmits a thorough examination guide explaining that a thorough examination is necessary, to the first terminal 10 together. Transmission of the various kinds of information from the second terminal 30 to the first terminal 10 may be performed via the server 20 or performed directly to the first terminal 10. Further, the medical institution and doctor selection unit 2052 of the server 20 can guide thorough examination means 40 for the patient to receive the thorough examination, based on the patient information (an address and the like).

The first terminal 10 displays the determination result by the doctor 60. When the determination result is that there is a suspected positive result, the thorough examination guide explaining that a thorough examination is necessary is displayed together (S13).

An operation in the case where the diagnosis result by the doctor 60 is that there is a suspected positive result will be explained below. If the diagnosis result by the doctor 60 is that there is a suspected positive result, the first terminal 10 displays the thorough examination guide explaining that a thorough examination is necessary, in addition to the determination result by the doctor 60 (S13).

If it is possible for the medical institution to which the doctor 60 belongs to perform the thorough examination, the thorough examination can be performed at the medical institution to which the doctor 60 belongs. In the case of the second terminal 30 transmitting the determination result that there is a suspected positive result, to the first terminal 10, the doctor 60 can additionally write that it is recommended to receive the thorough examination at the medical institution to which the doctor 60 belongs, to the thorough examination guide.

If it is not possible for the medical institution to which the doctor 60 belongs to perform the thorough examination, the thorough examination can be performed at the thorough examination means 40 specified by the doctor 60. Of course, the user 50 himself searches for thorough examination means 40 capable of performing the thorough examination so that the patient can receive the thorough examination at the thorough examination means 40.

In the case of performing the thorough examination at the medical institution to which the doctor 60 belongs or in the case of the thorough examination means 40 being connected to the server 20, the request information generation unit 2053 of the server 20 can transmit the determination result by the doctor 60 and the necessary patient information to the thorough examination means 40 via the disease information generation unit 202. The patient receives the thorough examination at the thorough examination means 40, and, at this time, the determination result by the doctor 60 and the necessary patient information can be used for the thorough examination (S41).

The thorough examination means 40 performs the thorough examination. When the thorough examination means 40 is connected to the server 20, a result of the thorough examination can be transmitted from the transmission unit 401 to the server 20. The server 20 receives the thorough examination result and can transmit the thorough examination result to the second terminal 30. When the thorough examination means 40 is connected to the server 20, the thorough examination means 40 can also transmit the thorough examination result to the first terminal 10 or communicate the thorough examination result to the patient not via the first terminal 10 but directly.

The doctor 60 can orally communicate a final diagnosis result in consideration of the thorough examination result to the patient. It is also possible for the doctor 60 to input the final diagnosis result in consideration of the thorough examination result to the second terminal 30 and transmit it to the first terminal 10 via the server 20. If the final diagnosis result in consideration of the thorough examination result is “positive”, the doctor 60 can orally communicate explanation of a treatment policy and the like to the patent together with the final diagnosis or can input it to the second terminal 30 and transmit it to the first terminal 10 via the server 20. The server 20 transmits the final diagnosis result in consideration of the thorough examination result and the treatment policy explanation to the first terminal 10. The first terminal 10 displays the final diagnosis result in consideration of the thorough examination result. When the final diagnosis result in consideration of the thorough examination result is “positive”, the first terminal 10 can display the explanation of the treatment policy by the doctor 60 and the like.

When the final diagnosis result in consideration of the thorough examination result is “positive”, a donation is sent to the first terminal 10 from the usage fees saved in the usage fee saving unit 2033 of the server 20 via the donation sending unit 2034. It is also possible for the first terminal 10 to request the donation from the server 20 through the donation request unit 107, or it is also possible for the donation sending unit 2034 of the server 20 to automatically send the donation to the user 50 who received the final diagnosis result of “positive”.

### REFERENCE SIGNS LIST

1 Biliary atresia diagnosis support management system
10 First terminal
20 Server
30 Second terminal
40 Thorough examination means
50 User
60 Doctor
101 Transmission/reception unit
102 User authentication unit
103 Usage fee sending unit
104 Donation reception unit
105 Information input unit
106 Disease information reception unit
107 Donation request unit
1051 Patient information input unit
1052 Stool image input unit
1053 Stool information input unit
1061 Determination result display unit
1062 Thorough examination guide display unit
1063 Treatment policy display unit
201 Transmission/reception unit
202 Disease information generation unit
203 Management unit
204 Determination unit
205 Doctor's determination request processing unit
2031 User account management unit
2032 Medical institution and doctor information management unit
2033 Usage fee saving unit
2034 Donation sending unit
2041 Similarity degree calculation unit
2042 Image accumulation unit
2043 Change analysis unit
2051 Doctor request decision unit
2052 Medical institution and doctor selection unit
2053 Request information generation unit
301 Transmission/reception unit
302 User authentication unit
303 Determination acceptance unit
304 Determination result input unit
305 Information request unit

1. A biliary atresia diagnosis support management system comprising a server,
the server comprising a transmission/reception unit receiving an image of stool from a living body; and
a determination unit determining whether biliary atresia is contracted or not from the image;
wherein the server further comprises:
a management unit comprising a usage fee saving unit saving received usage fees and a donation sending unit for sending a donation from the saved usage fees; and
a doctor's determination request processing unit requesting a determination by a doctor if the determination unit determines that there is a suspected positive result; and
if it is diagnosed that the living body needs treatment for biliary atresia as a result of a thorough examination, the server pays a donation for the treatment from the usage fees saved in the usage fee saving unit via the donation sending unit.

2. The biliary atresia diagnosis support management system according to claim 1, further comprising a first terminal,
the first terminal comprising a transmission/reception unit transmitting an image of stool from a living body and receiving a diagnosis result via the server;
a usage fee sending unit for paying the usage fee; and
a donation reception unit for receiving a donation.

3. The biliary atresia diagnosis support management system according to claim 2, wherein the first terminal further comprises a disease information reception unit comprising at least one means among a determination result display unit, a thorough examination guide display unit and a treatment policy display unit.

4. The biliary atresia diagnosis support management system according to claim 2, further comprising a second terminal transmitting at least any one of a determination result by the determination unit and a determination result by a doctor to at least any one of the first terminal and the server, wherein
if the determination unit determines that there is a suspected positive result, the second terminal transmits at least any one of the determination result by the doctor and a thorough examination guide to at least any one of the server and the first terminal.

5. A biliary atresia diagnosis support management system comprising:
(A) a first terminal for a user to transmit an image of stool of a newborn baby or an infant to a server using an application;
(B) a server receiving the image from the first terminal; and
(C) a second terminal transmitting at least any one of a determination result by a determination unit and a determination result by a doctor to at least any one of the first terminal and the server; wherein
the first terminal comprises a usage fee sending unit for the user of the application to pay a usage fee;
the server comprises:
a determination unit determining whether biliary atresia is contracted or not from the image;
a usage fee saving unit saving received usage fees; and
a donation sending unit for sending a donation from the saved usage fees;
if the determination unit determines that there is a suspected positive result, the server transmits a request for a determination by a doctor together with the determination result to the second terminal via the server;
if the determination unit determines that there is a suspected positive result, the second terminal transmits at least any one of the determination result by the doctor and a thorough examination guide to at least any one of the server and the first terminal; and
if it is diagnosed that the newborn baby or the infant needs treatment for biliary atresia as a result of a thorough examination, the server pays a donation for the treatment from the usage fees saved in the usage fee saving unit via the donation sending unit.

6. The biliary atresia diagnosis support management system according to claim 5, further comprising (D) thorough examination means for examining whether biliary atresia is contracted or not, wherein the thorough examination means performs the thorough examination about whether the newborn baby or the infant has contracted biliary atresia or not.

7. The biliary atresia diagnosis support management system according to claim 5, wherein the first terminal further comprises a donation request unit for the user to request the donation.

8. The biliary atresia diagnosis support management system according to claim 5, wherein, if the determination unit determines that biliary atresia is negative, the determination result of being negative is transmitted to the first terminal.

9. The biliary atresia diagnosis support management system according to claim 5, wherein
the determination unit further comprises a change analysis unit;
the change analysis unit analyzes a change between an image of stool of the newborn baby or the infant transmitted before and the image of stool of the newborn baby or the infant newly transmitted; and
the determination unit determines whether biliary atresia is contracted or not based on a result of the analysis.

10. The biliary atresia diagnosis support management system according to claim 5, wherein the first terminal further comprises a stool information input unit for transmitting stool information.

11. The biliary atresia diagnosis support management system according to claim 5, wherein the second terminal comprises an information request unit requesting further information from the server.

12. An application program for biliary atresia diagnosis to be installed on a terminal that is capable of transmitting an image of stool of a newborn baby or an infant to a server, the application program comprising:
means for a user of an application to send a usage fee;
means for causing information about a newborn baby or an infant to be inputted;
means for causing an image of stool of the newborn baby or the infant to be inputted;
means for receiving a guide for a thorough examination for examining whether biliary atresia is contracted or not;
means for displaying information about a determination result about a biliary atresia diagnosis received from the server or a doctor; and
means for receiving a donation for treatment for biliary atresia.

13. The biliary atresia diagnosis support management system according to claim 3, further comprising a second terminal transmitting at least any one of a determination result by the determination unit and a determination result by a doctor to at least any one of the first terminal and the server, wherein
if the determination unit determines that there is a suspected positive result, the second terminal transmits at least any one of the determination result by the doctor and a thorough examination guide to at least any one of the server and the first terminal.

14. The biliary atresia diagnosis support management system according to claim 6, wherein the first terminal further comprises a donation request unit for the user to request the donation.

15. The biliary atresia diagnosis support management system according to claim 6, wherein, if the determination unit determines that biliary atresia is negative, the determination result of being negative is transmitted to the first terminal.

16. The biliary atresia diagnosis support management system according to claim 7, wherein, if the determination unit determines that biliary atresia is negative, the determination result of being negative is transmitted to the first terminal.

17. The biliary atresia diagnosis support management system according to claim 6, wherein
the determination unit further comprises a change analysis unit;
the change analysis unit analyzes a change between an image of stool of the newborn baby or the infant transmitted before and the image of stool of the newborn baby or the infant newly transmitted; and
the determination unit determines whether biliary atresia is contracted or not based on a result of the analysis.

18. The biliary atresia diagnosis support management system according to claim 7, wherein
the determination unit further comprises a change analysis unit;
the change analysis unit analyzes a change between an image of stool of the newborn baby or the infant transmitted before and the image of stool of the newborn baby or the infant newly transmitted; and
the determination unit determines whether biliary atresia is contracted or not based on a result of the analysis.

19. The biliary atresia diagnosis support management system according to claim 8, wherein
the determination unit further comprises a change analysis unit;
the change analysis unit analyzes a change between an image of stool of the newborn baby or the infant transmitted before and the image of stool of the newborn baby or the infant newly transmitted; and
the determination unit determines whether biliary atresia is contracted or not based on a result of the analysis.

20. The biliary atresia diagnosis support management system according to claim 6, wherein the first terminal further comprises a stool information input unit for transmitting stool information.
